(19) United States
(12) Patent Application Publication
(10) Pub. No.: US 2011/0092799 A1
(43) Pub. Date: Apr. 21, 2011

(54) Active implant medical device (AMID) and medical imaging scanner communications involving patient-specific AIMD configuration

(75) Inventor: Michael Steckner, Richmond Heights, OH (US)

(73) Assignees: Kabushiki Kaisha Toshiba, Tokyo (JP); Toshiba Medical Systems Corporation, Otawara-shi (JP)

(21) Appl. No.: 12/662,448

(22) Filed: Apr. 19, 2010

Related U.S. Application Data
(63) Continuation-in-part of application No. 12/588,483, filed on Oct. 16, 2009.

(51) Int. Cl. A61B 5/055

(52) U.S. Cl. 600/411

(57) ABSTRACT

A communications link between an active implanted medical device (AIMD) and a magnetic resonance imaging (MRI) machine enables an information exchange including data relating to a configuration of a lead of the AIMD as installed in the patient and/or an operational parameter limit of the MRI machine determined from the data. The data relating to the configuration of the lead in the patient may include a size and/or position of an effective loop area enclosed by the lead. The data relating to the configuration of the lead in the patient may include data corresponding to a pictorial representation, such as an x-ray of the patient having the AIMD, of the lead and at least part of the patient's body. An operation of the MRI machine may be stopped or a warning/alarm initiated if the operational parameter limit (e.g., maximum power limit of a gradient or RF field) determined from the data relating to the configuration of the lead in the patient is exceeded.

### CROSS-REFERENCE TO RELATED APPLICATION

This application is a continuation-in-part (CIP) of application Ser. No. 12/588,483 filed Oct. 16, 2009, the entire content of which is hereby incorporated by reference in this application.

### BACKGROUND

1. Technical Field

Non-limiting, exemplary embodiments relate to an active implanted medical device (AIMD) for use in an environment of a magnetic resonance imaging (MRI) machine. More particularly, non-limiting, exemplary embodiments relate to a bi-directional communications link between the AIMD and the MRI machine which permits information to be exchanged to enable determination of an acceptable operation mode (e.g., whether operation is within “safe” operational limit(s)) for the AIMD and MRI machine, and to an apparatus having a MRI machine and an associated AIMD communications system.

Other non-limiting, exemplary embodiments relate to a communications link between the AIMD and the MRI machine which permits information such as data relating to an actual configuration of the AIMD in a patient (e.g., size and/or position of an effective loop area enclosed by the AIMD lead and/or an operational parameter limit determined based on the actual, patient-specific configuration of the AIMD) to be exchanged.

2. Description of Related Art

Active implanted medical devices (AIMDs) are widely used in human patients for a number of different medical applications. Examples of AIMDs include a cardiac pacemaker, a neurostimulator, a defibrillator, a hearing implant, a drug or insulin pump, etc. In the case of a cardiac pacemaker, for example, a small device having a housing and one or more wire leads extending from the housing is implanted under a human patient's skin. An integrated circuit or microprocessor located within the housing initiates electrical pulse signals transmitted through the wire leads to the human patient's heart to help control arrhythmias.

However, the environment produced by certain medical imaging scanners may be problematic for a patient having an AIMD. For example, a MRI system typically presents a hostile environment for AIMDs. In particular, the peak RF power of a MRI application (e.g., in the imaged volume) frequently exceeds many thousands of Watts. A high power RF field, such as that used in a MRI application, may therefore induce a very high voltage and/or current in the AIMD. This induced signal may cause the AIMD to operate improperly or may damage or even destroy the integrated circuit or microprocessor of the AIMD. Moreover, gradient magnetic fields produced by the MRI system may induce eddy currents in the housing or other component of the AIMD, thereby causing overheating of the AIMD. In certain circumstances, the combination of an AIMD in a MRI system can even be lethal to the patient. The current design of some AIMDs may therefore render them mutually exclusive with respect to certain MRI systems.

To resolve such problems, efforts are underway to improve the compatibility of medical imaging scanners and AIMDs. In particular, advances are being made in the design of AIMDs to make them “safer” for patients undergoing MRI scans. For example, concepts such as an AIMD broadcasting its operational limits, or even using RFID technology (e.g., see U.S. Patent Application Publication no. 2007/0257800 A1) to interrogate and broadcast device information, have been considered.

The degree of success in determining and reaching “safer” levels of operation may vary between different AIMDs. For example, a “safer” level of operation determined for a first AIMD in a particular medical imaging scanner may be different from a corresponding “safer” level determined for a second AIMD (e.g., an AIMD from a different manufacturer or a different AIMD model from the same manufacturer). Also, determination of these “safer” levels may have scanner specific implications.

Insofar as AIMDs and the medical imaging scanner might be mutually compatible at some level unique to the particular make and model of AIMD, it would be beneficial to consider ways to permit the AIMD and medical imaging scanner to communicate, in real time, important parameter limits defining the level of compatibility (e.g., operational parameter limits such as a limit on a location of the AIMD positioned in the medical imaging scanner) for the AIMD, and/or the medical imaging device prior to scanning. Since these parameter limits will be unique for a particular make and model of AIMD, it would also be beneficial to have the appropriate operational limits uniquely determined and accurately transcribed. This transcription would preferably not be performed by a human operator, and not subject to undue medical record inaccuracies.

In addition to providing real-time communications to define the level of compatibility for the AIMD and medical imaging scanner, it would also be beneficial to permit the AIMD and medical imaging scanner to conduct real-time communications to confirm that the AIMD senses no unusual or unacceptable conditions in the patient, such as an unacceptable monitored temperature of the patient. It may also be useful for the AIMD and/or medical imaging scanner to record data associated with a scanning event and relevant technical details for both operational, developmental and safety reasons.

Parameter limits defining the level of compatibility may differ even if the AIMDs of the same make and model are utilized. For example, the actual configuration of an AIMD within one patient may differ from the actual configuration of an AIMD, of the same make and model, within another patient. These differences in actual configuration may result from differences in the paths that are traveled by AIMD leads within respective patients. These differences in actual configuration may also result from differences in the size and/or position of an area approximately closed by a loop formed by the AIMD lead (hereinafter “loop area” or “lead area”) with another such area in a different patient.

The actual configuration of an AIMD in a particular patient thus has an effect on the level of compatibility between the AIMD and the medical imaging scanner. For example, the size of the loop area and/or the position of the loop area will affect the level of compatibility between the AIMD and the medical imaging scanner. The loop area will accumulate the power of the magnetic fields of the medical imaging scanner as a function of the size of the loop area. Namely, a larger loop area will collect more power from the medical imaging scanner.

The size and position of the loop area is patient specific—even AIMDs of the same make and model are utilized. That is, the size and location of the loop area are not fixed values from patient to patient, but are instead unique for a particular patient. The level of compatibility between the AIMD and medical imaging scanner may therefore differ from patient to patient even if the same type of AIMD is installed. These differences in compatibility levels can therefore be significant. The level of compatibility thus cannot be precisely determined prior to installation. In short, the unique installation of the AIMD into a particular patient results in a unique configuration (i.e., geometry) of the AIMD within the patient, which in turn results in a unique compatibility level with the medical imaging scanner.

It would therefore be beneficial to permit data relating to the actually installed configuration of the AIMD within a particular patient to be utilized when defining levels of compatibility between the installed AIMD and the medical imaging scanner. It would be further beneficial for the data relating to the actually installed configuration of the AIMD within a particular patient and/or level of compatibility defined therefrom to be exchanged readily between the AIMD and the medical imaging scanner. Moreover, it would be advantageous to store such configuration data in the AIMD even prior to the AIMD even beginning communications with the medical imaging scanner so that as such communications begin, the stored data is ready to be communicated and thus levels of compatibility that need to be defined can be done so quickly and efficiently.

### SUMMARY

Such problems are solved, in one non-limiting, exemplary embodiment, by creating a bi-directional communications link between an AIMD (e.g., a cardiac pacemaker) and a medical imaging scanner (e.g., a MRI machine) and using the bi-directional communications link to exchange information to determine an acceptable mode of operation for the AIMD and medical imaging scanner. In more detail, information may be exchanged between the AIMD and the medical imaging scanner to enable determination of whether or not operation of the AIMD and the medical imaging scanner satisfies “safe” operation levels. If so, a further operation of the medical imaging scanner may be enabled. If not, operation (or at least some functionality) of the medical imaging scanner and/or AIMD may be disabled. The information exchanged over the bi-directional communications link and subsequent processing of this information may therefore enhance patient safety and system performance during a scan performed by the medical imaging scanner. The exchanged information may be archived in the AIMD and/or medical imaging scanner to enable an update to patient history records, and to optimize future scan settings and/or future product development.

In another non-limiting, exemplary embodiment, an active implanted medical device (AIMD) for use with a magnetic resonance imaging (MRI) machine comprises: an integrated circuit, and a bi-directional communications interface operably coupled to the integrated circuit. The bi-directionally communications interface may communicate information with the MRI machine to determine whether the AIMD and the MRI are operating in an acceptable mode of operation. This determination may be empirically derived as a result of an actual operation of the AIMD with the MRI machine. This determination may include determining whether operation satisfies a limit on at least one operational parameter of the AIMD, the MRI machine and/or patient. The limit may relate to a limit on a location of the AIMD within an imaging bore of the MRI machine, or a limit on a power level of a magnetic field or a radio-frequency (RF) field of the MRI machine.

The AIMD may further comprise a sensor (e.g., a calibrated pick-up coil) for sensing a gradient magnetic field of the MRI machine. The sensor may be operably coupled to the integrated circuit which may determine a location of the AIMD within an imaging bore of the MRI machine based on the gradient magnetic field sensed by the sensor. The communications interface of the AIMD may communicate information relating to the determined location of the AIMD to the MRI machine. Alternatively, the communications interface of the AIMD may communicate information relating to the sensed gradient magnetic field to the MRI machine so that the MRI machine (as opposed to the AIMD itself) is able to determine a location of the AIMD within an imaging bore of the MRI machine based on the information sensed by the gradient magnetic field. The integrated circuit may determine whether a strength or exposure rate of the sensor to the gradient magnetic field sensed by the sensor is within an acceptable operational limit.

The AIMD may further comprise a sensor for sensing a RF field of the MRI machine. The RF sensor may be operably coupled to the integrated circuit which may determine whether a power level of or an exposure rate to the RF field sensed by the sensor is within an acceptable operational limit. Alternatively, the communications interface may communicate information relating to sensed RF field so that the MRI machine (as opposed to the AIMD itself) is able to determine whether a power level of or an exposure rate to the RF field sensed by the sensor is within an acceptable operational limit.

The AIMD may further comprise an alarm that produces an audible alarm signal when the limit is not satisfied. The integrated circuit may produce an alarm signal when an operational limit is not satisfied, and the communications interface may communicate information relating to the alarm signal to the MRI machine to disable operation of the MRI machine.

In another non-limiting, exemplary embodiment, an active implanted medical device (AIMD) for use with a magnetic resonance imaging (MRI) machine comprises a sensor that senses an operational parameter, and an integrated circuit operably coupled to the sensor, wherein the integrated circuit may generate an alarm signal when the integrated circuit determines that the sensed operational parameter forms an unacceptable condition. The unacceptable condition may be an unacceptable AIMD location. Alternatively or additionally, the sensed operational parameter may be a temperature of at least one component of the AIMD, and the unacceptable condition may be that the temperature exceeds a threshold level. Alternatively or additionally, the sensed operational parameter may be a change in a component of the AIMD, and the unacceptable condition may be that an amount of the change exceeds a threshold level. Alternatively or additionally, the sensed operational parameter may be a change in a characteristic of a patient in the MRI machine, and the unacceptable condition may be that an amount of the change exceeds a threshold level. Alternatively or additionally, the sensed operational parameter may be a level of exposure to a gradient magnetic field or a RF field of the MRI machine, and the unacceptable condition may be that the level of exposure exceeds a threshold level. Alternatively or additionally, the sensed operational parameter may be a rate of exposure to a gradient magnetic field or a RF field of the MRI machine, and the unacceptable condition may be that the rate of exposure exceeds a threshold level. The alarm signal may comprise an audible alarm signal. The AIMD may further comprise a bi-directional communications interface, operably coupled to the integrated circuit, that communicates information relating to the alarm signal to the MRI machine to disable an operation of the MRI machine. The AIMD may disable one or more of its own operations when the integrated circuit determines that the sensed operational parameter forms an unacceptable condition.

In another non-limiting, exemplary embodiment, a system comprises: a MRI system and a bi-directional AIMD communications interface operably coupled to the MRI system. The bi-directional AIMD communications interface may bi-directionally communicate information with an AIMD to determine whether the AIMD and the MRI system are operating in an acceptable mode of operation. This determination may be empirically derived as a result of an actual operation of the MRI system with the AIMD. This determination may include determining whether a limit on at least one operational parameter of the AIMD, the MRI system and/or patient has been satisfied. The limit may relate to a limit on a strength level of or exposure rate to a magnetic field of the MRI machine, or a limit on a power level of or an exposure rate to a radio-frequency (RF) field of the MRI system.

The limit may relate to a limit on a temperature of an AIMD component, change in a characteristic of an AIMD component and/or a patient characteristic. The limit may relate to a limit on a location of the AIMD within an imaging bore of the MRI system. The communicated information may include a disable signal for the MRI system. The MRI system may generate a disable signal based on the information received by the communications interface from the AIMD.

In another non-limiting, exemplary embodiment, a system comprises: a MRI system and a bi-directional AIMD communications interface operably coupled to the MRI system, wherein the bi-directional AIMD communications interface bi-directionally may communicate information with an AIMD to determine whether an operation of the MRI system should be enabled or disabled. The communicated information may include information relating to an alarm condition, and a determination may be made that the operation should be disabled based on the information relating to the alarm condition.

The communicated information may include information relating to the AIMD's unacceptable exposure to a magnetic field or a radio-frequency (RF) field of the MRI system, and a determination may be made that the operation should be disabled based on the information relating to the AIMD's unacceptable exposure to the magnetic field.

The communicated information may include information relating to the AIMD's unacceptable exposure to a radio-frequency (RF) field of the MRI system, and a determination may be made that the operation should be disabled based on the information relating to the AIMD's unacceptable exposure to the RF field. Alternatively or additionally, the communicated information may include information relating to the AIMD's unacceptable location within an imaging bore of the MRI machine, and a determination may be made that the operation should be disabled based on the information relating to the AIMD's unacceptable location. Alternatively or additionally, the communicated information may include information relating to an unacceptable temperature of at least one component of the AIMD, and a determination may be made that the operation should be disabled based on the information relating to the unacceptable temperature. Alternatively or additionally, the communicated information may include information relating to an unacceptable change in at least one component of the AIMD, and a determination may be made that the operation should be disabled based on the information relating to the unacceptable change. Alternatively or additionally, the communicated information may include information relating to an unacceptable change in a characteristic of a patient, and a determination may be made that the operation should be disabled based on the information relating to the unacceptable change.

The communicated information may include information relating to a triggering signal provided by the AIMD, and the MRI system may determine that the operation should be enabled by the triggering signal. The triggering signal may be determined based on physiological state of a patient. The physiological state of the patient may relate to the patient's QRST complex.

In another non-limiting, exemplary embodiment, a method of operating a system comprising a MRI machine and a bi-directional AIMD communications interface disposed to bi-directionally communicate with an AIMD bi-directionally communicates information with the AIMD, processes information received from the AIMD as at least part of the bi-directionally communicated information, and determines whether the AIMD and the MRI machine are operating in an acceptable mode of operation based on the processed information.

The acceptable mode of operation may be empirically derived as a result of an operation of the MRI machine with the AIMD. Determining the acceptable mode of operation may include determining whether a limit on at least one operational parameter of the AIMD, the MRI machine and/or a patient has been satisfied. The limit may relate to a limit on a strength level of or an exposure rate to a magnetic field of the MRI machine, a limit on a power level of or an exposure rate to a radio-frequency (RF) of the MRI machine, a limit on a location of the AIMD within an imaging bore of the MRI machine, a limit on a temperature of at least one component of the AIMD, a limit on a change in at least one component of the AIMD, a limit on a change in a characteristic of a patient.

The received information may relate to a location of the AIMD within an imaging bore of the MRI machine determined based on the gradient magnetic field of the MRI machine. The MRI system (as opposed to the AIMD itself) may determine a location of the AIMD within an imaging bore of the MRI machine based on the information received by the communications interface from the AIMD.

In another non-limiting, exemplary embodiment, a method of operating a system comprising a MRI machine and a bi-directional AIMD communications interface disposed to bi-directionally communicate with an AIMD receives information from the AIMD using the bi-directional AIMD communications interface, the received information indicating an unacceptable operating condition, processes the received information, and disables operation of the MRI machine based on the processed information. The unacceptable operating condition may relate to the AIMD's overexposure to a magnetic field or a radio-frequency (RF) field of the MRI machine. Alternatively or additionally, the unacceptable operating condition may relate to the AIMD's location within an imaging bore of the MRI machine. Alternatively or additionally, the unacceptable operating condition may relate to a temperature of at least one component of the AIMD. Alternatively or additionally, the unacceptable operating condition may relate to a change in at least one component of the AIMD. Alternatively or additionally, the unacceptable operating condition may relate to a change in a characteristic of a patient.

In another non-limiting, exemplary embodiment, a method of operating a system comprising a MRI machine and at least one bi-directional AIMD communications interface disposed to bi-directionally communicate with an AIMD receives information from at least one AIMD using the bi-directional AIMD communications interface, the received information including a triggering signal for operation of the MRI machine, processes the received information, and enables a MRI operation of the MRI machine based on the triggering signal. The received information may relate to a physiological state of a patient, and enabling the MRI operation may include coordinating the MRI operation with the patient's physiological state. The physiological state of the patient may relate to the patient's QRST complex.

In another non-limiting, exemplary embodiment, a method of operating a system having a MRI machine and a bi-directional AIMD communications interface bi-directionally communicates information with an AIMD using the AIMD communications interface, processes information received by the AIMD communications interface in the communicated information, and determines whether to enable or disable an operation of the MRI machine based on the processed information. The operation of the MRI machine may be disabled if no communication of information between the AIMD and the AIMD communications interface is detected for predetermined period of time. The communicated information may include information relating to a communications protocol such that subsequent communicated information does not include confidential data.

In another non-limiting, exemplary embodiment, a system comprises (i) a MRI system, and (ii) an AIMD communications interface, operably coupled to the MRI system, that communicates information with an AIMD having a lead, the communicated information including data relating to a configuration of the lead in a patient. The lead of the AIMD may define a loop area, and the data relating to the configuration of the lead in the patient may include a size and/or position of the loop area. The data relating to the configuration of the lead in the patient may include data corresponding to a pictorial representation (such as an x-ray) of the lead and at least part of a body of the patient. The information received by the AIMD communications interface from the AIMD may include information indicating a limit on at least one operational parameter of the MRI machine determined by the AIMD based on the data relating to the configuration of the lead in the patient. Instead of the AIMD determining the limit, the MRI machine may determine the limit based on the data relating to a configuration of the lead in the patient. The limit may relate to a maximum limit on a strength level of a magnetic field and/or a radio-frequency (RF) field of the MRI machine.

In another non-limiting, exemplary embodiment, a method of operating a system comprising a MRI machine and an AIMD communications interface disposed to communicate with an AIMD having a lead comprises (i) communicating information with the AIMD; and (ii) processing information received from the AIMD as at least part of the communicated information, the communicated information including data relating to a configuration of the lead in a patient. A lead of the AIMD may define a loop area, and the data relating to the configuration of the lead in the patient may include a size and/or position of the loop area. The data relating to the configuration of the lead in the patient may include data corresponding to a pictorial representation (such as an x-ray) of the lead and at least part of a body of the patient. The communicated information received from the AIMD may include information indicating a limit on at least one operational parameter of the MRI machine determined by the AIMD based on the data relating to the configuration of the lead in the patient. Instead of the AIMD determining the limit, the MRI machine may determine the limit based on the data relating to the configuration of the lead in the patient. The limit may relate to a maximum limit on a strength level of a magnetic field and/or a radio-frequency (RF) field of the MRI machine.

In another non-limiting, exemplary embodiment, an active implanted medical device (AIMD), having at least one lead for use with a magnetic resonance imaging (MRI) machine, comprises (i) an integrated circuit that processes data relating to a configuration of the lead in a patient; and (ii) a communications interface, operably coupled to the integrated circuit, that communicates information with the MRI machine, the communicated information including the data relating to a configuration of the lead in the patient and/or a limit on at least one operational parameter of the MRI machine determined based on the data relating to the configuration of the lead in the patient. The lead of the AIMD may define a loop area, and the data relating to the configuration of the lead in the patient may include a size and/or position of the loop area. The data relating to the configuration of the lead in the patient may include data corresponding to a pictorial representation (such as an x-ray) of the lead and at least part of a body of the patient. The communicated information may include data relating to the limit, the limit being determined by the integrated circuit of the AIMD based on the data relating to the configuration of the lead in the patient and communicated information received from the MRI machine. The communicated information received by the communications interface of the AIMD from the MRI machine may include information indicating the limit, the limit being determined by the MRI machine based on the data relating to the configuration of the lead in the patient. The limit may relate to a maximum limit on a strength level of a magnetic field and/or a radio-frequency (RF) field of the MRI machine.

### BRIEF DESCRIPTION OF THE DRAWINGS

These and other advantages of the exemplary embodiments will be more completely understood and appreciated by careful study of the following more detailed description in conjunction with the accompanying drawings, in which:

FIG. 1 is a schematic view of an exemplary AIMD system in accordance with one non-limiting, exemplary embodiment;

FIG. 2 is an overall system-wide schematic view of an exemplary system incorporating a magnetic resonance imaging (MRI) machine and an AIMD system in accordance with one non-limiting, exemplary embodiment;

FIG. 3 is an exemplary diagram showing, for example, an imaging bore of the MRI machine illustrated in FIG. 2;

FIG. 4 is a flowchart depicting an exemplary method of operating the system illustrated in FIGS. 1-3;

FIG. 5 is a schematic view of an exemplary AIMD system in accordance with another non-limiting, exemplary embodiment;

FIGS. 6A-6B are diagrams showing a part of the AIMD system of FIG. 5 and a loop area formed by a lead of the AIMD system; and

FIG. 7 is a flowchart depicting an exemplary method of operating the system illustrated in FIGS. 2-3 and 5-6B.

### DETAILED DESCRIPTION OF NON-LIMITING, EXEMPLARY EMBODIMENTS

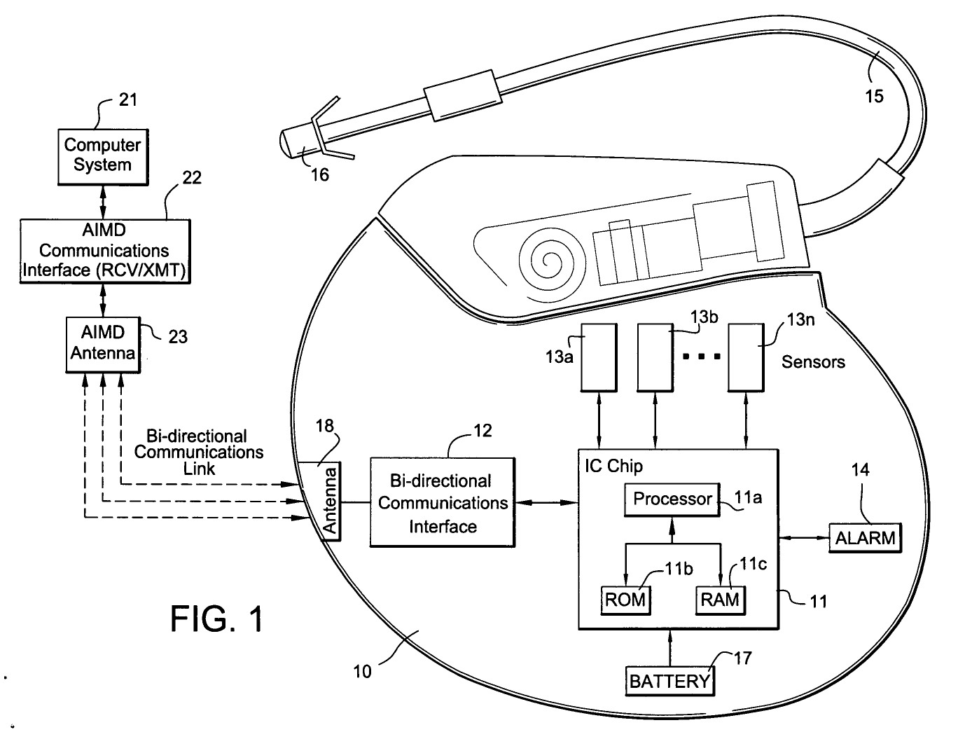

FIG. 1 illustrates an exemplary AIMD system which may be used in accordance with one non-limiting, exemplary embodiment. The AIMD system includes an AIMD 10 which communicates bi-directionally (e.g., to specifically coordinate MRI/AIMD activities in the hostile MRI environment) with a MRI system receiving component including a computer system 21, an AIMD communications interface 22, an AIMD antenna 23. The computer system 21 may be one of a system of computers which control MRI system functions as will be discussed below. Alternatively, the computer system 21 may be a separate additional computer system which communicates with a MRI system computer.

While the MRI system is described in detail below, it will be understood that the computer system 21 may be part of or communicate with an alternative medical imaging scanner system such as, but not limited to, an x-ray system or a CT scanning system. Also, while FIG. 1 illustrates a cardiac pacemaker as the AIMD 10, it will be understood that another type of AIMD may be alternatively used in the AIMD system. Examples of these other types of AIMDs include, but are not limited to, a neurostimulator, a defibrillator, a hearing implant, and an implantable drug or insulin pump, as well as any accessory to these examples.

The AIMD communications interface 22 and the AIMD 10 may establish a wireless, bi-directional communications link. Through this bi-directional communications link, the AIMD communications interface 22 exchanges (i.e., both transmits and receives) information via its corresponding AIMD antenna 23 with the AIMD 10. The exchanged information may include for example, the AIMD's or MRI system's identification information (e.g., model, manufacturer, hardware/software version information), the AIMD's or the MRI system operational limits, detected operational parameters, patient information, identification of potentially hostile devices or environments that may interact with a particular AIMD, MRI system and/or patient, communications protocol information governing subsequent communications and information enabling a recipient device to perform a programming function. Alternatively, the bi-directional communications link may exchange information specifically and exclusively for coordinating the MRI/AIMD activities (e.g., exchanging the AIMD's or MRI system's identification information, the AIMD's or the MRI system operational limits, detected operational parameters, patient information, identification of potentially hostile devices or environments that may interact with a particular AIMD, MRI system and/or patient), with the other information such as information enabling a recipient device to perform a programming function being communicated over another bi-directional pathway.

The AIMD 10 may include an IC chip 11, a bi-directional communications interface 12, sensors 13a-13n, an alarm 14, a lead 15, an electrode 16, a battery 17 and an antenna 18. A housing (typically made of a metal material) may enclose the IC chip 11, communications interface 12, one or more of the sensors 13a-13n, the alarm 14, the battery 17 and the antenna 18. However, one or more of the sensors 13a-13n, the alarm 14 and the antenna may be located partially or wholly outside of housing (e.g., mounted on the outer surface of the housing itself). An inductive loop may be placed on the outside of the AIMD housing so that the battery 17 may be recharged via the inductive loop's interaction with the gradient magnetic fields of the MRI system.

The IC chip 11 includes a processor 11a and memory (ROM 11b and RAM 11c as shown in FIG. 1) for storing data and executable control instructions. The processor 11a of the IC chip 11 executes the control instructions to perform various functions such as receiving, retrieving, writing, processing and transmitting data. The data stored in the memory 11b-11c of the IC chip 11 may include, for example, patient information such as identification and medical history information, identification of any MRI machine that would potentially present a hostile environment for the AIMD 10 and/or patient, device history or service record information, device identification information and configuration data. The memory 11b-11c of the IC chip 11 may archive any information received from the MRI system such as any information describing steps, actions or parameters of the MRI system or any information sensed by the sensors 13a-13n.

The data stored in the memory 11b-11c of the IC chip 11 may be encrypted via standard techniques to prevent unauthorized tampering. Also, the data stored in the memory 11b-11c of the IC chip 11 may include a communications protocol governing future communications with the MRI system. For example, the communications protocol, once communicated and enacted by the AIMD 10 and MRI system, may govern communications over the bi-directional communications link to ensure that the AIMD 10 does not transmit any confidential patient information or ensure that the AIMD 10 in the MRI system meets certain expectations.

The processor 11a of the AIMD 10 may disable certain operational AIMD function(s) that may interfere with the operation of the MRI system. For example, the processor 11a may require the AIMD 10 to enter a passive minimal operating condition until notice is provided that a particular procedure is completely or partially finished, and/or may require some further communication prior to performing further AIMD operation (e.g., between MRI scans). The AIMD 10 and MRI system could then perform a “handshake” to confirm that both devices are ready for further operation. Disabling one or more of the operational AIMD function(s) may be triggered, for example, by one or more of the operational parameters sensed by sensors 13a-13n being outside of acceptable limits.

The antenna 18 of the AIMD may be made of a conductive material and is tuned to the same carrier frequency of the AIMD antenna 23. The carrier frequency is set so that communications over the bi-directional communications link does not interfere with other operations of the AIMD 10 and/or the MRI system. During normal operation, IC chip 11 may modulate or demodulate the carrier field from AIMD communications interface 22 in order to retrieve and/or transmit data from/to AIMD communications interface 22. The data transmitted back to AIMD communications interface 22 may then be communicated to computer system 21.

The lead 15 comprises a wire inserted into a patient's vein or artery so that the electrode 16 is located in the patient's heart. The lead 15 may send electrical impulses generated by the AIMD 10 to the patient's heart via electrode 16 to prompt the heart to beat at a normal rate. While only a single lead 15 is illustrated in FIG. 1, it will be understood that multiple leads leading to different portions of the patient's heart may extend from the housing of the AIMD 10.

The sensors 13a-13n may detect various operational parameters of the AIMD 10, MRI system and/or patient. For example, at least one sensor 13a-13n may be utilized to detect a radio frequency (RF) field emitted by the MRI system, a gradient magnetic field emitted by the MRI system, a static magnetic field emitted by the MRI system, a temperature of one or more components (the case, lead 15 and/or electrode 16) of the AIMD 10, changes in the leads, and/or characteristics of the patient including heart rate, QRST complex, heart beat and/or changes in patient characteristics (e.g., tissue damage and/or changes in patient temperature). While FIG. 1 illustrates each of the sensors 13a-13n located within the housing of the AIMD 10, it will be understood that one or more of these sensors can be outside of the housing. For example, any one of the sensors may be incorporated into the lead 15 and/or electrode 16. It will also be understood that any one of the sensors 13a-13n could be utilized to measure more than one operational parameter and that more or less sensors than that illustrated in FIG. 1 may be present depending on the number of the parameters that need to be monitored.

As one example, one or more of the sensors 13a-13n may comprise a small pick-up coil for detecting the strength of the magnetic field of the MRI system. The pick-up coil can be calibrated so as to produce an induced signal corresponding to the strength of the magnetic field. Due to the gradient magnetic field emitted by the MRI system, different locations within the MRI imaging bore will experience different magnetic field strengths. Accordingly, the induced signal from the calibrated pick-up coil will differ based upon the different magnetic field strengths at the different locations within the MRI imaging bore. The IC chip 11 may utilize the induced signal (e.g., the amplitude of the induced signal) and its calibration to determine the position of the AIMD 10 within the imaging bore of the MRI system. That is, the IC chip 11 may utilize the induced signal of the calibrated pick-up coil to determine a relative distance estimate of the AIMD 10 within the MRI system imaging bore (e.g., distance from the center of the imaging bore). Based on the determined location of the AIMD 10, the IC chip 11 can then determine whether or not the AIMD 10 is within a permitted location. A determination can thus be made whether or not the AIMD is within an acceptable region (or outside of unacceptable region) of the MRI imaging bore based upon the magnetic field strength detected by the calibrated pick-up coil. This can be particularly useful for those MRI scans where multiple positions of the patient's body are imaged. Each determined position of the AIMD 10 may be verified for compliance with acceptable ranges. The AIMD operations may thus be limited inclusively or exclusively to certain areas within the imaging bore of the MRI system.

As another example, one or more of the sensors 13a-13n may detect the RF field emitted by the MRI system. Based upon the detected RF field, the IC chip 11 may determine the AIMD's and/or patient's level of exposure to the RF field and/or the exposure rate to the RF field. The IC chip 11 may then determine whether the power level or exposure rate of the AIMD 10 to the RF field is within an operational limit of the AIMD 10. In particular, the IC chip 11 may calculate an exposure level or exposure rate of the AIMD 10 to the RF field, and compare either or both of these parameters to respective operational limits or thresholds. If, for example, the exposure level and/or rate of exposure to the RF field exceeds an operational limit (e.g., predicted dosimetry threshold), further processing such as initiating an alarm to alert medical personnel and/or automatically stopping operation of the MRI system and/or AIMD functionality may be performed. The AIMD 10 can thus independently compute its own dosimetry and then determine whether dosimetry is within acceptable operational limits. Alternatively, the AIMD 10 may communicate its own dosimetry to the MRI system via its bi-directional communications interface 12 and antenna 18 to enable the MRI system (as opposed to the AIMD 10 itself) to determine whether the AIMD's dosimetry is within acceptable limits. The MRI system can then, for example, disable a MRI process if the computed dosimetry does not satisfy the acceptable limits. The communication of the AIMD's dosimetry information to the MRI system and the MRI system's processing of the received dosimetry information may be performed in real-time, thereby providing immediate notification to an operator of the MRI system. In addition to communicating information regarding the AIMD's actual dosimetry, the operational limits and/or ranges utilized to compare with the actual dosimetry to determine whether or not the AIMD 10 and/or patient is experiencing an unacceptable level of exposure or exposure rate to the RF field may also be communicated. It will be understood that while the sensor described in the foregoing discussion determines whether the exposure level or exposure rate of the AIMD 10 exceeds dosimetric limits for RF fields, the sensor could alternatively be utilized to determine whether or not there is an unacceptable exposure or exposure rate to a magnetic (e.g., gradient and/or static) field or whether or not any other sensed parameter is at an unacceptable level.

As another example, both the AIMD 10 and the MRI system can make a determination of whether operation is within acceptable limits. The results of these determinations may be then compared with each other, thereby creating a fail-safe check. For example, a MRI process may be disabled if the determinations by the AIMD 10 and the MRI system are different from each other, or alternatively, a MRI process may be disabled only if both determinations by the AIMD 10 and the MRI system indicate operation outside of acceptable limits.

As yet another alternative example, one or more sensors 13a-13n may detect a patient's physiological state (e.g., QRST complex and/or heart rate). As with the other detected operational parameters, these states can be compared with acceptable operational limits or ranges to determine whether an unacceptable operating mode or condition is present. Also, the IC chip 11 may utilize the patient information to provide a triggering signal to the MRI system in order to coordinate the actions of the MRI system with the patient's physiological state (e.g., the patient's QRST complex). The triggering signal generated by the IC chip 11 is transmitted to the MRI system via the bi-directional communications interface 12 and antenna 18 to enable operation of the MRI system.

The alarm 14 may generate an audible alarm if an acceptable mode of operation of the AIMD 10 and/or MRI system is not present. For example, if the IC chip 11 determines that one or more of the operational parameters detected by one or more of the sensors 13a-13n is outside of its respective acceptable operational limits or ranges, the alarm 14 may generate an audible alarm to provide notification of an unacceptable condition to the MRI system operator or other medical personnel. For example, the alarm 14 may generate an audible alarm if one or more of the AIMD's sensors 13a-13n detects a magnetic or RF field that is too strong, one or more components of the AIMD 10 is overheating, and/or one or more of characteristics of the AIMD 10, patient or MRI system changes too much. Alternatively, the alarm may provide a signal indicating that one or more of the operational parameters sensed by one or more of sensors 13a-13n is acceptable. That is, the alarm 14 may indicate an acceptable mode of operation (rather than an unacceptable mode).

After hearing the audible alarm produced from alarm 14, the MRI system operator may manually disable one or more functions of the MRI system. Alternatively, the patient may hear the alarm and let the MRI system operator know (e.g., by squeezing an air driven squeeze bulb) to disable the MRI process, thereby enabling the patient to be part of the stopping process. As yet another alternative, the IC chip 11 may communicate, in real time, a disable signal via the bi-directional communications interface 12 and antenna 18 to the MRI system when an unacceptable mode of operation has been determined. The MRI system may thus be automatically disabled via the signal communicated from the AIMD 10 via its bi-directional communications interface 12 and antenna 18. The MRI system may therefore immediately stop its operation.

Rather than the AIMD 10 producing the alarm signal, the AIMD 10 can communicate the relevant information (e.g., the operational parameters detected by sensors 13a-13n and optionally the corresponding operational limits or ranges) to the MRI system so that the MRI system (rather than the AIMD 10 itself) can determine whether or not an acceptable mode of operation exists. If an acceptable mode of operation does not exist, the MRI system may then automatically disable one or more (or even all) of its operations and/or provide an audible or visual alarm signal for the MRI system operator to manually disable operation(s).

The IC chip 11 may also continually confirm that the bi-directional communications link between the AIMD 10 and the MRI system is operative. For example, the IC chip 11 of the AIMD 10 may determine whether or not the AIMD 10 and MRI system are communicating with each other for a predetermined interval (e.g., the AIMD 10 and MRI system are “pinging” each other every 10 milliseconds or another regular time period). If the IC chip 11 does not confirm that the communications link is operative, the IC chip 11 may generate an alarm signal for alarm 14 to emit an audible alarm, or communicate the generated alarm signal through the bi-directional communications interface 12 to the MRI system so that the MRI system can generate an audible or visual alarm or immediately disable one or more operations of the MRI system. As another example, the MRI system (rather than the AIMD 10) may make a determination that the bi-directional communications link is no longer operative. Based upon this determination, the MRI system may disable one or more of its own functions automatically or provide notification to the MRI system operator.

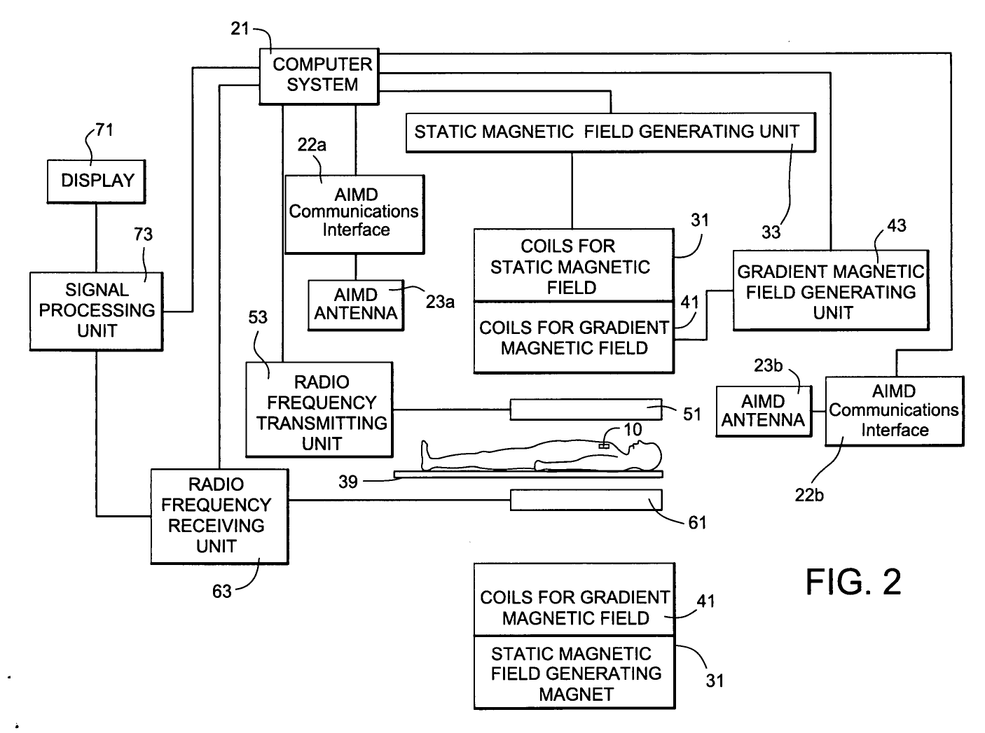

FIG. 2 is a general overview of major components of an exemplary system including a MRI system and an AIMD system. The AIMD system in this exemplary system may be implemented by the AIMD system illustrated in FIG. 1. The MRI system includes static magnetic field coils 31, gradient magnetic field coils 41, a RF transmitting coil 51 (or an array of RF transmitting coils) and RF receiving coils 61. A computer system 21 controls gradient magnetic field coils 41, RF transmitting coil 51 and RF receiving coils 61 through respective units 43, 53 and 63 (and may in some circumstances have some control associated with static magnetic field coils 31 via unit 33). The computer system 21 also communicates with signal processing unit 73 which is capable of generating a display resulting from a MRI application on display 71.

The static magnetic field coils 31 generate a powerful (e.g., 0.5T, 1.5T or 3.0T) uniform magnetic field. The gradient magnetic field coils 41 emit gradient magnetic fields in three orthogonal directions upon receiving appropriate outputs from gradient magnetic field generating unit 43. A RF transmitting coil emits a RF field through operation of the radio frequency transmitting unit 53 to excite nuclei of patient tissue to NMR in the imaged volume. The frequency of the RF field emitted from the RF transmitting coil 51 may have a frequency F0 equal to, for example, 63.6 MHz or 127 MHz. The particular frequency F0 used is determined in large part by the strength of the static uniform magnetic field. RF receiving coils 61 receive RF NMR response signals from NMR patient tissue nuclei. The signal processing unit 73 utilizes the received NMR RF signals to generate an image to be displayed on display 71.

In addition to the MRI system, the system illustrated in FIG. 2 includes an implementation of the AIMD system illustrated in FIG. 1. In particular, the system includes two AIMD communications interfaces 22a, 22b each having a respective AIMD antenna 23a, 23b. Each of the AIMD communications interfaces 22a, 22b are connected to computer system 21. While the system illustrated in FIG. 1 includes two AIMD communications interfaces 22a, 22b, those of ordinary skill in the art will understand that only one or more than two AIMD communications interfaces can be utilized depending on the extent of the AIMD operating range needed. The AIMD detection range needed may merely overlap with the MRI imaging tunnel (approximately 1-2 meters in length) in which MRI imaging typically occurs or extend to nearby areas or well beyond.

The communications interface 22a, 22b and the AIMD 10 are tuned to the same operating frequency. The AIMD communications interfaces 22a, 22b establish a bi-directional communications link with the AIMD 10 for exchanging information via its corresponding AIMD antennas 23a, 23b. As described above, the exchanged information may include for example, the AIMD's or MRI system's identification information (e.g., model, manufacturer, hardware/software version information), the AIMD's or the MRI system operational limits, detected operational parameters, patient information, identification of potentially hostile devices or environments that may interact with a particular AIMD, MRI system and/or patient, communications protocol information governing subsequent communications, and information enabling a recipient device to perform a programming function.

The data stored in the computer system 21 may be encrypted via standard techniques to prevent unauthorized tampering. Also, the data stored in computer system 21 may include a communications protocol governing future communications with the AIMD 10. For example, the communications protocol, once communicated and enacted by the AIMD 10 and MRI system, may govern communications over the bi-directional communications link to ensure that the AIMD 10 does not transmit any confidential patient information or may ensure that the AIMD 10 in the MRI system meets certain expectations. The computer system 21 may also store any of the data received from the AIMD 10.

The computer system 21 may disable one or more (or even all) of operational functions of the MRI system. For example, upon receiving a signal from the AIMD 10 indicating that the MRI system, AIMD 10 and/or patient is experiencing an unacceptable mode of operation, the computer system 21 may automatically and immediately stop one or more (or even all) operations of the MRI system. Alternatively, the computer system 21 may, upon receiving a signal from the AIMD 10 indicating that the MRI system, AIMD 10 and/or patient is experiencing an unacceptable mode of operation, the computer system 21 may provide an audible and/or visual (e.g., via display 71) notification to the MRI system operator to manually disable one or more (or even all) operations of the MRI system.

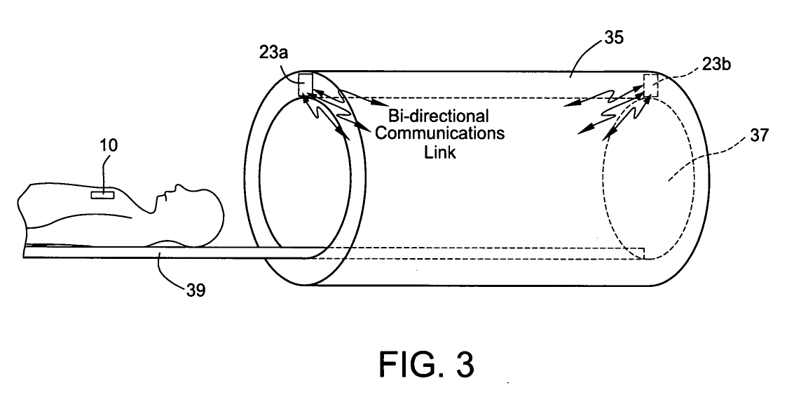

FIG. 3 shows one exemplary implementation of certain components of the system illustrated in FIG. 2. In particular, the static magnetic field coils 31 in this embodiment are shaped in a cylindrical form 35. Cylindrical form 35 defines an imaging bore or tunnel 37 into which a patient may be slid via table 39. AIMD antennas 23a and 23b may be attached to a portion of cylindrical form 35.

When an AIMD 10 is within the communication range of one or more of the AIMD antennas 23a, 23b, a wireless, bi-directional communications link with the AIMD 10 may be established. The AIMD communication range may include the entire or only part of the imaging area defined by bore 37 and areas nearby. The AIMD communication range would thus overlap at least with the static magnetic, gradient magnetic and RF fields of the MRI system.

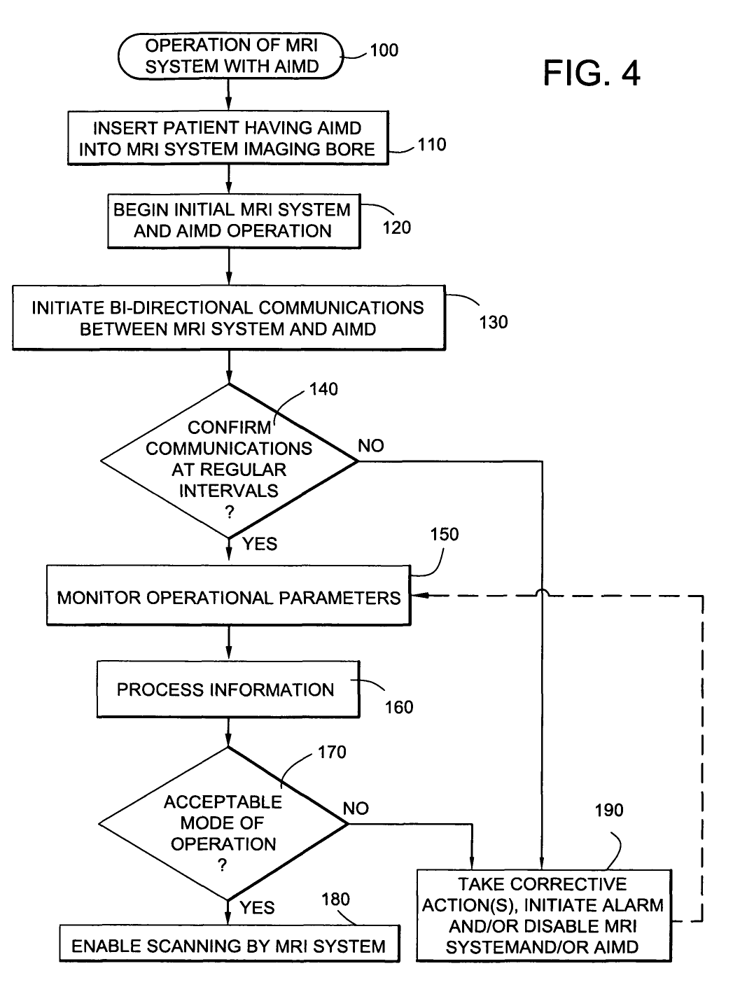

FIG. 4 illustrates a non-limiting exemplary process 100 which may be performed using the system illustrated in FIGS. 1-3. Through this process, whether an acceptable mode of operation for the MRI system and the AIMD can be detected. For example, whether the MRI system and/or the AIMD operates within at least one operational parameter limit may be determined. If so, a MRI system operation (e.g., a MRI scanning operation of the patient) can be initiated under the conditions of the acceptable mode of operation and/or a signal by the AIMD or the MRI system may be generated to indicate acceptable operation. If not, an alarm may be generated by the MRI system or the AIMD itself to alert a MRI system operator to manually stop operation of the MRI system, or operation of the MRI system and/or AIMD may be automatically stopped. Determining whether the MRI system and/or AIMD operates within at least one operational parameter limit may involve, for example, determining whether the AIMD's exposure level or rate to a magnetic field (e.g., gradient or static magnetic field) or RF field is within a predetermined limit, whether the AIMD's location is acceptably within (or outside of) a particular part of the imaging volume of the MRI system, whether the AIMD (or at least one of its components) is within a predetermined temperature range and/or whether a change of a monitored characteristic of the AIMD is within an acceptable range. Monitored parameters of the patient, such as a heart rate, QRST complex, temperature or changes in any of these parameters, which may indicate that the patient would not be a good candidate for a MRI scan, may also be utilized to at least temporarily stop operation of the MRI system and/or AIMD 10, prevent another operation from even beginning, or initiate an alarm at the MRI system and/or AIMD 10.

Before beginning operation of the MRI system, a patient having the AIMD 10 is moved into the imaging bore or tunnel 37 of the MRI system (Step 110). For example, a patient having the AIMD 10 is slid via table 39 into the cylindrical form 35 as illustrated in FIGS. 2-3. The communication range of the AIMD 10 communication interfaces 23a, 23b may be set to cover the entire MRI imaging bore such that the bi-directional communications link between the AIMD 10 and the MRI system can be established whenever the AIMD 10 is positioned in the MRI imaging bore and/or its nearby area.

The computer system 21 then begins an initial MRI system operation concurrently with on-going operation of the AIMD 10 (step 120). The initial MRI system operation may be a “full” MRI scanning process. However, this initial MRI system operation may alternatively comprise less than all of the MRI system functions. For example, the computer system 21 may drive the RF transmitting coil 52 without driving the gradient magnetic field coils 41, particularly in the case where the major concern is the possible overexposure of the AIMD 10 to the RF field.

The AIMD 10 and the MRI system may then establish a wireless, bi-directional communications link (step 130). Those skilled in the art will appreciate that the order of steps 110-130 are not fixed. For example, the patient may be placed on the table 39 (part of step 110), the bi-directional communications between the MRI system and AIMD established (step 130), and then the patient inserted into the bore 37 (part of step 110).

As discussed above, the bi-directional communications link between the AIMD 10 and the MRI system enables information to be exchanged, to among other things, enable a determination to be made regarding whether or not the AIMD 10 and/or MRI system is operating acceptably. The bi-directional communications link between the MRI system and the AIMD 10 may be established by one of the devices sending a suitable request to establish the communications link, and the other device acknowledging and accepting the request. Bi-directional communications can thus be performed between the interfaces 12 and 22 via antennas 18 and 23.

One of the initial pieces of information that may be communicated between the MRI system and the AIMD 10 is a communications protocol for governing subsequent communications. This protocol may determine the format for data representation, signaling, authentication and error detection for successful communication over the bi-directional communications link. As one example, the communications protocol may define limits or restrictions on the data that may be communicated, such as restricting or preventing any communication of confidential patient information over the communications link.

The AIMD 10 and/or MRI system (e.g., the computer system 21) may monitor the communications over the bi-directional communications link. Through this monitoring, the AIMD 10 and/or MRI system can confirm that the bi-directional communications link is still operative. For example, a confirmation can be continually made to confirm whether or not the AIMD 10 and the MRI system are “pinging” each other at regular intervals (e.g., pinging each other every 10 milliseconds—although those skilled in the art will recognize that this regular interval may be set in a predetermined manner to another time period). If the communications link is not confirmed to be operative (“NO” in step 140), an alarm may be initiated by the AIMD 10 and/or MRI system (step 190). This alarm will provide audio or visual notice to the MRI system operator that some adjustment is needed for the MRI system and/or AIMD 10. Alternatively or additionally, the MRI system and/or AIMD 10 may be automatically and immediately disabled, or corrective actions may be automatically made by the MRI system and/or AIMD or made by the MRI system operator (step 190).

Sensors 13a-13n may detect various operational parameters of the AIMD 10, MRI system and/or the patient (step 150). As discussed above, the detected operational parameters may include, for example, the RF field emitted by the MRI system, the gradient magnetic field emitted by the MRI system, the static magnetic field emitted by the MRI system, temperature characteristics of any component of the AIMD 10 (e.g., the AIMD's case, leads or electrodes), patient characteristics, and any changes in the above-mentioned operational parameters.

The sensors 13a-13n may provide information relating to the detected operational parameters to the IC chip 11. The IC chip 11 processes the received information (step 160). The processing may include communicating the received information to the MRI system via the AIMD's communications interface 12 and antenna 18 for further processing. This processing (by the IC chip 11 and/or the computer system 21) may include comparing the detected operational parameters to corresponding operational limits (e.g., an upper limit and/or lower limit) to determine whether or not the detected operational parameters indicate an acceptable operation. For example but without limitation, the power level or exposure rate of the AIMD 10 to the RF field emitted by the MRI system (as detected by one or more of the sensors 13a-13n) may be compared to an upper power level or exposure rate limit. Alternatively, the strength or exposure rate of the AIMD 10 to a magnetic field (gradient or static magnetic field) emitted by the MRI system (as detected by one or more of the sensors 13a-13n) may be compared to an upper limit to determine whether the AIMD 10 has been overexposed to the magnetic field. As yet further examples, the detected temperature of an AIMD 10 component (e.g., case, lead 15 or electrode 16) can be compared to a corresponding operational limit to determine whether any of these components is overheating. Changes in any of the operational parameters detected by sensors 13a-13n can also be compared to a particular operational limit to determine an unacceptable mode of operation. Typically, a wide change in a detected operational parameter (e.g., a great change in the tissue characteristics of a patient or in any component of the AIMD 10) may indicate an unacceptable mode of operation.

As a final example, the location of the AIMD 10 can be compared to corresponding limit(s) to determine whether the AIMD 10 is included within an acceptable range or excluded from an unacceptable range of positions within the MRI system. As described above, one or more of the sensors 13a-13n may include a pick-up coil which is calibrated to produce a induced signal having a particular amplitude based upon the strength of the magnetic field detected by the pick-up coil. The induced signal may be processed by the IC chip 11 to determine the AIMD's location within the MRI imaging bore. Comparison to a corresponding limit may thus enable a determination to be made regarding whether or not the AIMD 10 is located at an acceptable position.

The processing of information performed in step 160 may be entirely performed by the IC chip 11 of the AIMD 10. Alternatively, the “raw” data provided by the sensors 13a-13n can merely be forwarded by the IC chip 11 to the MRI system over the bi-directional communications link during its processing so that the computer system 21 (rather than the IC chip 11) may perform the further processing of step 160. The required processing may thus be divided between the IC chip 11 and the computer system 21 in any ratio. Also, not only may the data corresponding to the detected operational parameters be communicated over the bi-directional communications link, but the operational limits utilized to compare to the detected operational parameters may also be communicated over the bi-directional communications link.

The IC chip 11 and/or computer system 21 thus determines whether an acceptable mode of operation has been achieved (step 170). As discussed in detail above, this may be accomplished by determining whether or not at least one detected operational parameter satisfies its corresponding operational limit. The acceptable mode of operation can be defined in any manner on the basis of these comparison(s). For example, an unacceptable mode of operation can be determined (“NO” in step 170) if only one detected operational parameter does not satisfy its corresponding operational limit. Alternatively, an unacceptable mode of operation can be defined such that more than one operational parameter must fail to satisfy its corresponding operational limit.

If an unacceptable mode of operation is determined (“NO” in step 170), the IC chip 11 may generate a signal to activate the alarm 14 (step 190). This alarm 14 may be an audio signal which alerts the MRI system operator (and patient) of the unacceptable mode of operation. The MRI system operator can then take the required steps to resolve any potential problem resulting from the unacceptable mode of operation. Instead of (or in addition to) providing the generated signal to activate alarm 14, the IC chip 11 may transmit the generated alarm signal to the computer system 21 over the bi-directional communications link. The computer system 21 can thus provide an audio or visual notification to the MRI system operator of the unacceptable mode of operation or automatically disable MRI system operation(s) based on the transmitted alarm signal. Based upon this notification, the MRI system operator can take the required corrective step(s) to resolve any problem, including manually disabling the MRI system if necessary (step 190). Alternatively, the MRI system or AIMD 10 may be programmed to take corrective action to automatically resolve any problem (step 190). If at least an attempt of corrective action is manually and/or automatically made to resolve the problem (e.g., adjust an operational parameter of the AIMD and/or MRI system so that it will now satisfy a corresponding limit), then the operation can flow back to step 150 in a repeated loop manner (e.g., at least for a predetermined number of times) as indicated by the dashed lined illustrated in FIG. 4 (e.g., to monitor and determine if the now-adjusted parameter provides an acceptable mode of operation in steps 150-170).

In addition to generating and sending the alarm signal to the alarm 14, the IC chip 11 may automatically disable one or more functions of the AIMD 10 itself. The IC chip 11 may thus transmit a “disable” signal to the MRI system 21 over the bi-directional communications link to initiate an alarm and/or automatically disable the MRI system from one or more of its operations. The disabling of the MRI system and/or AIMD 10 (step 190) thus provides an emergency stop—even in the situation where the AIMD 10 and the MRI system have previously satisfied all operational limits.

Instead of transmitting a disable-trigger signal, “raw” data sensed by sensors 13a-13n and/or corresponding limits may be communicated to the computer system 21. In this case, the computer system 21 may determine that there is an unacceptable mode of operation. If so, the computer system 21 can provide the audio or visual alarm to the MRI system operator, automatically disable the MRI system itself, and/or provide a signal to the AIMD 10 to activate its alarm 14 and/or stop one or more of its functions.

If an acceptable mode of operation is determined (“YES” in step 170), further operation by the MRI system is permitted (step 180). For example, the computer system 21 may enable the MRI system to conduct a “full” scan. The scanning by the MRI system may utilize the same settings as determined when determining the acceptable mode of operation. The acceptable mode of operation may thus be uniquely and empirically determined using the same AIMD 10 and MRI system and their acceptable operating conditions which are later used in order to perform the “full” MRI scan. Potential harm to the AIMD 10 and/or patient can thus be minimized. The scanning by the MRI system may be triggered by the signal from the AIMD 10 and may be coordinated to data (e.g., patient data such as the patient's QRST complex or heartbeat) from the AIMD 10.

In addition to enabling MRI system operation (step 180) if an acceptable mode operation has been determined (“yes” in step 170), the alarm 14 may emit a signal to indicate the acceptable mode of operation. That is, the alarm 14 may be utilized to provide notice of acceptable mode of operation, rather than an unacceptable mode of operation. The sound of the alarm 14 indicating the acceptable mode of operation may be different than the sound indicating an unacceptable mode.

The process described in FIG. 4 may thus provide a more fail-safe system operation so that a MRI application may begin only if acceptable mode of operation is determined (e.g., at least one detected operational parameter satisfies its corresponding operational parameter limit). The computer system 21 will not permit the MRI scan from even beginning if an unacceptable mode of operation is determined (e.g., at least one detected operational parameter does not satisfy its corresponding operational parameter limit). If a MRI scan has already begun and an unacceptable mode of operation is determined, the computer system 21 can automatically and immediately stop the MRI operation or provide notice to the MRI system operator of the unacceptable mode of operation. Alternatively, if an unacceptable mode of operation is determined, an alarm signal from the AIMD 10 or the MRI system can provide notice to the patient and/or MRI system operation. Based on this notice, the MRI system operator may manually disable MRI operated based on his/her discretion and judgement.

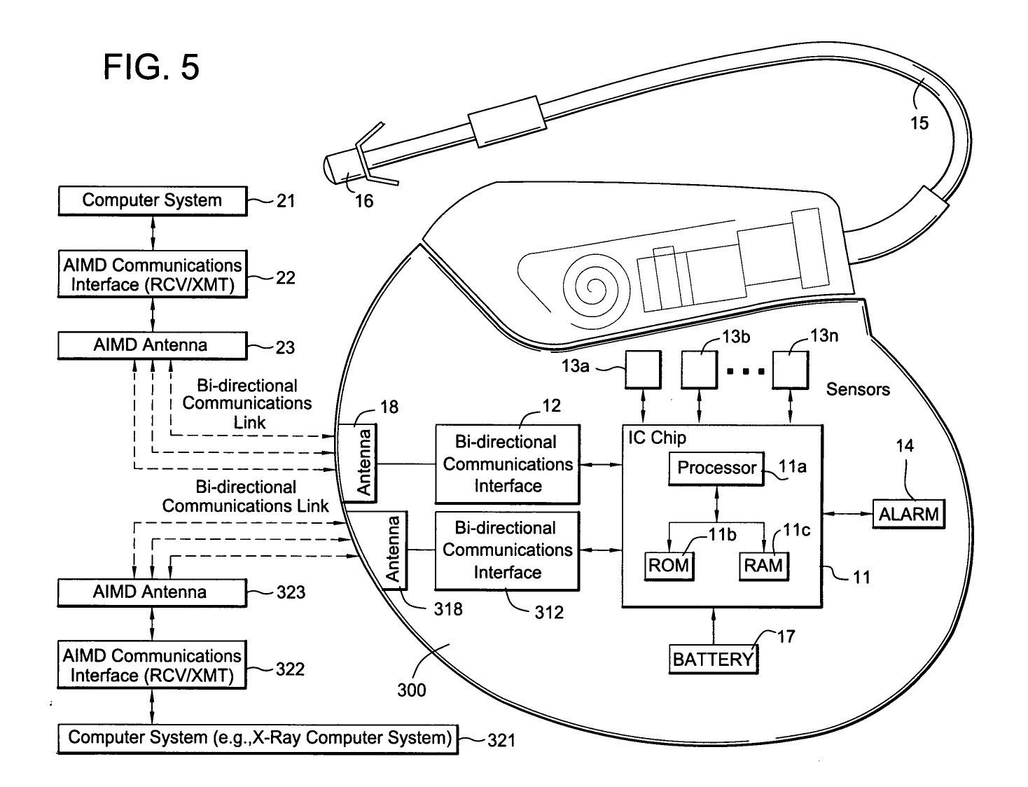

FIG. 5 illustrates an AIMD system which may be used in accordance with another non-limiting, exemplary embodiment. The AIMD system illustrated in FIG. 5 includes all of the structure and functionality of the AIMD system illustrated in FIG. 1. Like the AIMD system of FIG. 1, the AIMD system of FIG. 5 may be used in conjunction with the MRI system illustrated by FIGS. 2-3 and may perform all of the functionality illustrated in FIG. 4. Reference numerals in FIG. 5 which are duplicated with respect to FIG. 1 denote the identical structure and are capable of performing the identical functions and operation. Only differences between the AIMD system illustrated in FIG. 5 from the AIMD system illustrated in FIG. 1 will be discussed in more detail below.

The AIMD 300 illustrated in FIG. 5 includes all of the structure and is capable of performing all of the operation and functionality of the AIMD 10 illustrated in FIG. 1. Additionally, the AIMD 300 may include a second bi-directional communications interface 312 and a second antenna 318. Through the bi-directional communications interface 312 and the antenna 318, the AIMD 300 is capable of establishing a second bi-directional communications link. This second bi-directional communications link may be independent from the bi-directional communications link that the AIMD 300 may establish with computer system 21.

The AIMD system of FIG. 5 also includes a second AIMD antenna 323, a second AIMD communications interface 322 and a second computer system 321. The computer system 321 may be one or more of a system of computers which control a medical imaging scanner such as, but not limited to, an x-ray system or a CT scanning system, or even another MRI system. Also, while FIG. 5 illustrates a cardiac pacemaker as the AIMD 300, it will be understood that any other type of AIMD may alternatively be used in the AIMD system illustrated in FIG. 5, particularly any AIMD having a lead.

The antenna 318 of the AIMD 300 may be made of a conductive material and may be tuned to the same carrier frequency of the AIMD antenna 323. The carrier frequency is set so that communications over the second bi-directional communications link does not interfere with operations between the AIMD 10, the MRI system, the computer system 321 and/or communications over the other bi-directional communications link. During normal operation, the IC chip 11 may modulate or demodulate the carrier field from the AIMD communications interface 322 in order to exchange data with the AIMD communications interface 322. The data transmitted to the AIMD communications interface 322 may then be communicated to the computer system 321.

The bi-directional communications link established between the AIMD 300 and system 321 (via interfaces 312 and 322 and antennas 318 and 323) may be a wireless, bi-directional communications link. Through this bi-directional communications link, the system 321 may wirelessly exchange (i.e., transmit and/or receive) with the AIMD 300. The exchanged information may include, for example, data corresponding to the AIMD configuration (i.e., geometry) as actually installed in a patient or other body such as a phantom. The data relating to the configuration of the AIMD 300 as actually installed within a patient may include, for example, the size and/or position of the loop area approximately enclosed by the lead 15. The data relating to the configuration of the AIMD 300 as installed within the patient may additionally or alternatively correspond to a pictorial representation of the lead 15 (including its lead) and at least part of the body of the patient. This pictorial representation may be formed by an x-ray picture of the AIMD and its lead as actually installed within the patient and/or a simple digital representation of at least part of the patient's body, the AIMD and the AIMD's lead location. The exchanged information may additionally or alternatively include, for example, the AIMD's and/or the MRI system's operational limits—as determined from the AIMD configuration data within the patient. Other exchanged information over the bi-directional communications link established between the computer system 321 and the AIMD 300 may include the AIMD's 300 and/or the computer system's 321 identification information (e.g., model/manufacturer, hardware/software version information), detected operational parameters, patient information, identification of potentially hostile devices or environments that may interact with a particular AIMD system, computer system 321 and/or patient, or communications protocol governing subsequent communications and information enabling a recipient device to perform a programming function.

As noted above, the IC chip 11 includes a processor 11a and memory (ROM 11b and RAM 11c) for storing data and executable control instructions. The processor 11a of the IC chip 11 is capable of executing the control instructions to perform various functions such as receiving, retrieving, writing, processing and transmitting data. For example, the processor 11a is capable of executing control instructions to transmit/receive data to/from the computer system 321. Any data received by the AIMD 300 from computer system 321 may be written into and later accessed from the memory 11b or 11c. The data stored in the memory 11b, 11c of the IC chip 11, including the data received from computer system 321, may be encrypted via standard techniques to prevent unauthorized tampering. The data received and stored by the AIMD 300 from the computer system 321 may include data corresponding to the actually-installed configuration of the AIMD 300 (such as the actually-installed configuration of the AIMD lead 15) within a patient. This received and stored data may specifically be, for example, the loop size and/or position of the loop area approximately enclosed by the lead 15 as actually installed within a patient (e.g., as calculated by the computer system 321), and/or a pictorial representation (e.g., x-ray picture) of at least part of the patient body, the AIMD (including its lead and enclosed lead area) and/or its AIMD location. Rather than an x-ray picture which may have a great deal of extraneous information, the digital representation may simply indicate where a particular landmark of the patient's body is along with the representation of the AIMD 300 including its lead 15. The processor 11a or other device such as the computer system 21 may determine the loop size and/or position based on the pictorial representation or other digital representation received from the computer system 321.

The data that is received from the computer system 321 and later stored by the AIMD 300 in its memory may later be accessed by the processor 11a in order to determine for example the loop area size and/or position, and/or an operational parameter limit on the AIMD or MRI system. Additionally or alternatively, the processor 11a may access the data received from computer system 321 and stored in its memory 11b, and/or 11c and transmit this data to the computer system 21 so that the MRI machine can determine the loop area size and/or position, and/or an operational limit on itself or on the AIMD. These operational limits may include, for example, the maximum power levels of the RF or gradient fields.

The processor 11a of the AIMD 300 may disable certain operational AIMD function(s) that may interfere with the operation of the MRI system. Disabling one or more of the AIMD function(s) may be triggered, for example, by one or more of the operational parameters sensed by sensors 13a-13n being outside of acceptable limits that are defined on the basis of the patient-specific AIMD configuration data received from the computer system 321.

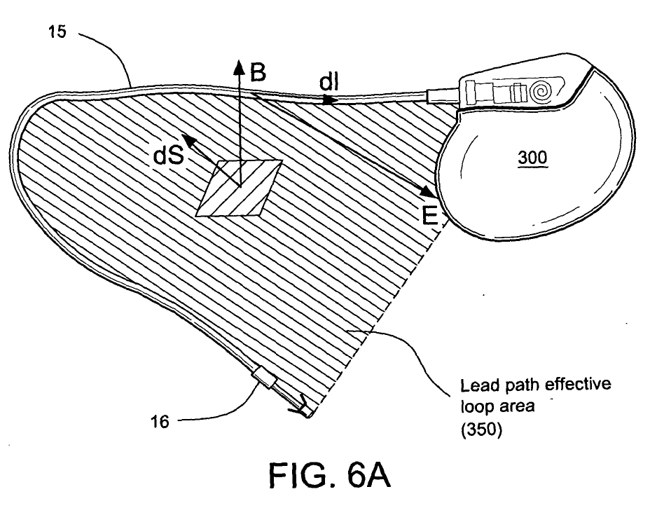

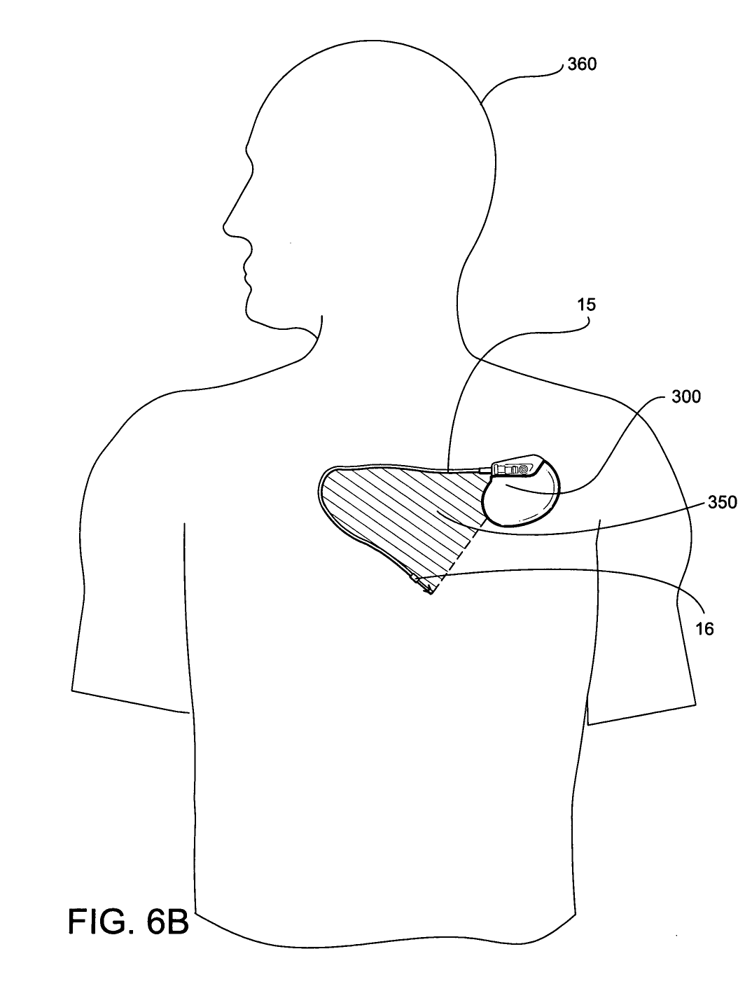

As illustrated in FIGS. 6A-6B, the lead 15 of the AIMD 300 comprises a wire inserted in a patient 360. (See also FIG. 2 illustrating an AIMD within a patient.) While only a single lead 15 is illustrated in FIGS. 5-6B, it will be understood that multiple leads leading to different portions of the patient may extend from the housing of the AIMD 300. Also, it is possible for a patient's AIMD to be removed and a new AIMD, including a new set of leads, put into the patient. In this situation, the old leads may remain in the patient, but are considered abandoned. Since these non-removed leads may present safety concerns, the existence, location and/or enclosed area of these non-removed leads may be tracked and stored in a database (e.g., a database of the computer system 21).

The following description discusses a single loop area effectively defined by a single lead 15. However, those skilled in the art will understand that the following discussion may be extended to cover each of the respective loop areas defined respectively by a plurality of leads (e.g., including abandoned leads of an otherwise removed AIMD).

The lead 15 of the AIMD 300 installed within a patient 360 (or phantom) encloses an approximately closed loop area 350. The dashed line illustrated in FIGS. 6A-6B shows where the loop area 350 is artificially closed off by the lead 15. In FIG. 6A, a small incremental area dS of the loop area 350 is graphically illustrated for mathematical concerns. In FIG. 6A, “B” represents a magnetic field, “d1” represents a small incremental length of the lead 15, and “E” represents an electric field or a change in voltage.

As illustrated in FIG. 6B, the lead 15 defines a loop area 350 having a specific size and position within the patient 360. However, if this same type (same make and model) of AIMD were installed in another patient, the size and/or position of the loop area may be different. That is, the configuration of an AIMD, including its lead, will be patient specific even if AIMDs of the same make and model are installed in these respective patients. The level of compatibility (e.g., operational parameter limits such the maximum permitted gradient and/or RF field strength from the MRI system illustrated in FIGS. 2-3) may differ from patient to patient due to the unique AIMD and AIMD lead installations within the patients. These levels of compatibility can therefore not be determined exactly prior to installation of the AIMD within the patient. Each installation of an AIMD into a particular patient presents a unique configuration, and thus may result in a unique compatibility determination.

The present example embodiments of FIGS. 5-6B allow for data corresponding to the actually-installed configuration of the AIMD 300 within a particular patient 360 to be utilized when defining the levels of compatibility between the installed AIMD 300 and the MRI system illustrated in FIGS. 2-3. To accomplish this, the computer system 321 is utilized to obtain the unique configuration (i.e., geometry) data of the AIMD (and in particular the AIMD's lead 15) as actually installed within the patient 360. For example, the computer system 321 may be an x-ray system which generates an x-ray picture data of the patient 360 having the already installed AIMD 300.

As described above, one or more of sensors 13a-13n may be calibrated so as to produce an induced signal corresponding to the strength of the magnetic field. Due to the gradient magnetic field emitted by the MRI system, different locations within the MRI imaging bore will experience different magnetic field strengths. The induced signal from one or more sensors 13a-13n will differ based on the different magnetic field strengths at the different locations within the MRI imaging bore. The IC chip 11 may utilize the induced signal (i.e., the amplitude of the induced signal) and an associated calibration to determine the position of the AIMD 300 within the imaging bore of the MRI system. Accordingly, the position of the AIMD 300, and hence the proximate position of the loop area of the AIMD 300 defined by its lead 15 within the imaging bore of the MRI system can be determined. Based on this determined location and/or other data corresponding to the configuration of the lead such as the size of the loop area, the processor 11 a of the AIMD 300 or the computer system 21 may determine an operational limit (such as maximum permitted power level of the gradient and/or RF fields) of the AIMD or MRI system.

In addition to the IC chip 11 continually confirming that the bi-directional communications link between the AIMD 300 and the MRI system is operative, the IC chip 11 may also continually confirm that the bi-directional communications link between the AIMD 300 and the computer system 321 is operative. In short, the IC chip 11 may continually confirm that either of the bi-directional communications link is still operative. For example, the IC chip 11 of the AIMD 300 may determine whether or not the AIMD 300 and the computer system 321 are communicating with each other for a predetermined interval (e.g., the AIMD 300 and the computer system 321 are “pinging” each other every 10 milliseconds or another regular time interval). If the IC chip 11 does not confirm that the communications link with the computer system 321 is operative, the IC chip 11 may generate an alarm signal for alarm 14 to emit an audible alarm, or communicate the generated alarm signal through the bi-directional communications interface 312 to the computer system 321 so that the computer system 321 can generate an appropriate audio or visual alarm. As another example, the computer system 321 (rather than the AIMD 300) may make a determination that the bi-directional communications link with the AIMD 300 is no longer operative. Based upon this determination, the computer system 321 may disable functionality automatically will provide notification to the operator of the computer system 321.

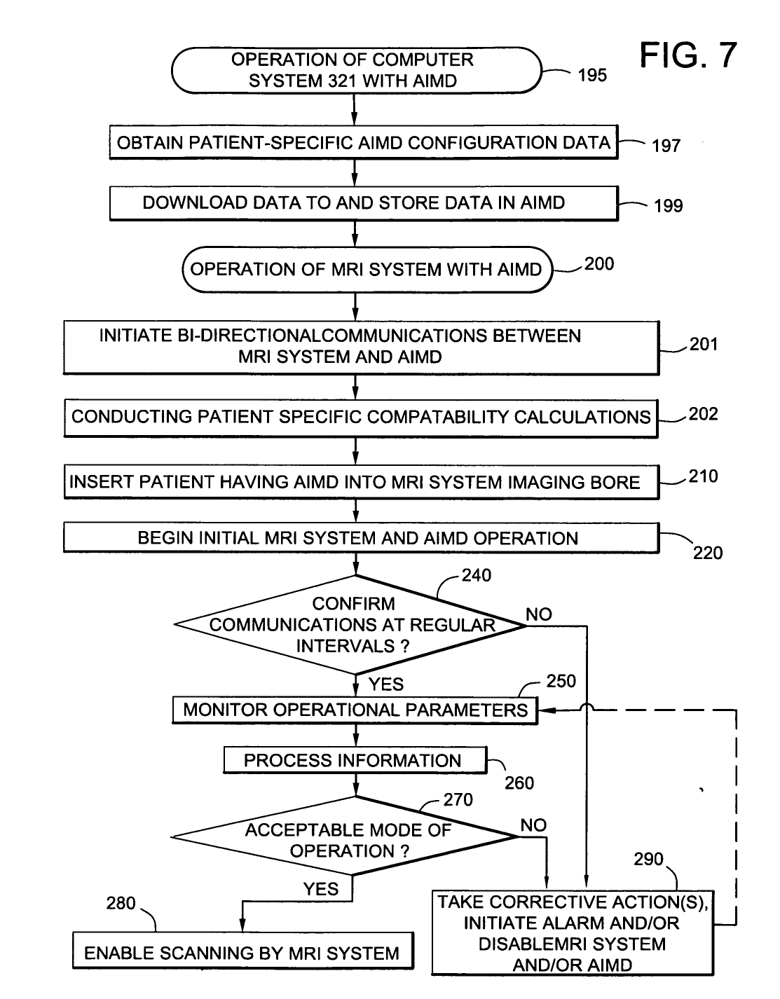

FIG. 7 illustrates a non-limiting exemplary process which may be performed using the system illustrated in FIGS. 2-3 and 5-6B. The exemplary process includes two main sub-processes: operation of the computer system 321 with the AIMD (sub-process 195 including steps 197 and 199)) and operation of the MRI system with the AIMD (sub-process 200 including steps 201-290). With respect to sub-process 200, steps 210-290 correspond essentially identically with steps 110-190, respectively, of the process 100 illustrated in FIG. 4. That is, step 210 corresponds essentially identically to step 110, step 220 corresponds to step 120, and so on. Only differences of steps 210-290 from their counterpart steps 110-190 will be discussed in detail. One significant difference is that the information that is processed in the sub-process 200 (steps 210-290) includes information relating to the specific AIMD configuration (e.g., lead configuration) as installed within a particular patient and operational limits (e.g., to determine whether an acceptable mode of operation is being accomplished) determined from the patient-specific AIMD configuration data.

As discussed above, the order of steps 110-130 of FIG. 4 are not fixed. Similarly, the steps indentified by reference numbers 201 (which corresponds to the step 130 of FIGS. 4), 210 and 220 are also not fixed and can be reordered.

As illustrated in FIG. 7, sub-process 195 (“Operation of Computer System 321 with AIMD”) includes step 197 (“Obtain Patient-Specific AIMD Configuration Data”). Each installation of an AIMD within a particular patient is a unique installation and thus presents a unique configuration of the AIMD within that patient. This is the case even if the same type of AIMD (same make and model) is installed in different patients. After the AIMD 300 is installed in the patient 360, the computer system 321 obtains configuration data of the AIMD 300 installed in the patient 360. For example, the computer system 321 may be an x-ray system or other medical imaging scanner to obtain a representation of the AIMD 300 within the patient 360. The obtained data may include a complete x-ray picture of the actual loop area 350 of the AIMD lead 15 or some other representation of this area. For minimizing memory requirements and required computations, the obtained data may comprise a simple digital representation of part of the patient's body, the AIMD 300 and its lead location. A complete representation of the patient's body is not necessary and may provide a great detail of extraneous information. Accordingly, only a component of the patient's body such as a landmark feature easily identifiable in the representation might be required—so long as the actual loop area and its size and position can be determined.

After the patient-specific configuration data is obtained (step 197), the data can then be downloaded and stored in the AIMD 300 (step 199 of sub-process 195). For example, an inductive system can be utilized to wirelessly transmit over the bi-directional communications link between the AIMD 300 and the computer system 321 the data obtained in step 197 to the AIMD 300. This data may then be stored in the memory 11b and/or 11c of the IC chip 11. The processor 11a may then access this data from the memory 11b, 11c for future processing including, for example, calculation of the actual size and/or position of the loop area. As an alternative, the data received and stored by the AIMD 300 from the computer system 321 may include data indicating the loop size/position—as already calculated by the computer system 321.

In the sub-process 200, the AIMD 300 and the MRI system may establish a wireless, bi-directional communications link (step 201—counterpart to step 130 of FIG. 4). Again, the order of steps 201, 210 and 220 can be reordered. This bi-directional communications link between the AIMD 300 and the MRI system enables information to be exchanged, to for example enable a determination regarding whether or not the AIMD 300 and/or MRI system is operating acceptably (e.g., within operational limits determined from the data generated by the computer system 321 and downloaded to and stored by the AIMD 300).

One of the initial pieces of information that may be communicated between the MRI system and the AIMD 300 is the data corresponding to the unique AIMD/patient installation characteristics obtained by the system 321 and downloaded to and stored by the AIMD 300. For example, the x-ray picture data or other digital representation of the AIMD 300 and its lead 15 as actually installed in the patient may be transmitted from the AIMD 300 to the computer system 21 in order for the computer system 21 to calculate the size and/or position of the AIMD lead loop area 350, and then to determine an operation limit on the MRI system or AIMD 300. Alternatively, the computer system 21 can download the entire gradient and RF waveforms to the AIMD 300 so that the AIMD 300 (rather than the computer system 21) can perform all needed calculations to determine an operational limit. Once calculated by the AIMD 300, the AIMD 300 can then transmit the calculated limit to the computer system 21.

Early exchange of the information between the computer system 21 and the AIMD 300 enables patient specific compatibility calculations to be conducted relatively early (step 202). These calculations may include a determination of the size and/or position of the loop area by either of the AIMD 300 or the computer system 21. While step 202 is depicted in FIG. 7 as occurring after step 201 and before steps 210 and 220, those skilled in the art will understand that this step 202 may alternatively be performed after 210 or 220. As yet another alternative, certain patient specific compatibility calculations such as the size and/or position of the loop area may be determined by an executed algorithm stored in the AIMD 300 (or even computer system 321 can then communicated to AIMD 300) even before the sub-process 200 begins. Calculating the loop area prior to sub-process 200 by the AIMD 300 itself (rather than the computer system 21), provides the advantage of enabling the results of these calculations to be accessed at a relatively early point in time.

As part of step 202, an operational limit to the AIMD and/or MRI system may be determined based on the exchanged data. In particular, either the computer system 21 or the processor of the AIMD 300 may determine an operational parameter limit on the AIMD and/or MRI system based on the data originating from the computer system 321. For example, given the magnitude and time rate of change of the magnetic field of the MRI system, the algorithm (executed by either of the computer system 21 or the AIMD 300) may compute an induced voltage. The magnitude of the magnetic field is a function of position, and the time rate of change of the magnetic field is a function of the imposed pulse sequence of the MRI system. Some nominal “worst case” magnitude and time rate of change of the magnetic field can be chosen, and then along with the size and/or position of the loop area (as determined from the data originating from the computer system 321 and downloaded to and stored by the AIMD in sub-process 195), the algorithm may generate maximum permissive operational limits on the AIMD and/or MRI system. As yet one example, the AIMD 300 or the computer system 21 may execute the algorithm to compute a maximum permitted power level of the RF and/or gradient magnetic fields of the MRI system based on the size and/or position of the loop area and other parameters. As the size and/or position of the loop area changes, these maximum permitted power levels may change as well.

As described above, steps 240-290 of FIG. 7 correspond to steps 140-190 of FIG. 4, respectively. In step 260, the processing (by the IC chip 11 and/or the computer system 21) may include comparing the detected operational parameters to corresponding operational limits (e.g., comparison using the maximum permitted power level of the gradient and/or RF fields determined based on the patient-specific AIMD configuration data such as the size/position of the loop area) to determine whether or not the detected operational parameters indicate an acceptable operation. For example, but without limitation, the power level or exposure rate of the AIMD 300 to the RF field emitted by the MRI system (as detected by one or more of the sensors 13a-13n) may be compared to the upper power level or exposure rate limit of the RF field (as determined from the executed algorithm using the patient-specific AIMD configuration data originating from the system 321, including the size/position of the loop area). Alternatively, the strength or exposure rate of the AIMD 300 to a magnetic field (gradient or static magnetic field) emitted by the MRI system (as detected by one or more of the sensors 13a-13n) may be compared to an upper limit (as determined from the executed algorithm using the patient-specific AIMD configuration data originating from the system 321, including the size/position of the loop area). If the actual power level or exposure rate of the RF or magnetic field exceeds the determined upper limits, an unacceptable mode of operation (“NO” in step 270). In this case, a corrective action may be taken, or an alarm at the AIMD 300 and/or computer system 21 may be initiated, and/or the MRI system and/or AIMD 300 may be disabled automatically or based upon system operator intervention (step 290; see the above description with respect to step 190 which is the counterpart of step 290).

The example embodiment of FIG. 7 (in conjunction with the system illustrated in FIGS. 2-3 and 5-6B) thus permits data relating to the actually installed configuration of the AIMD within a particular patient (e.g., size/position of loop area 350 of the actually installed AIMD 300) to be utilized when defining levels of compatibility (e.g., maximum permitted power levels of the RF and magnetic fields) between the actually installed AIMD 300 and the MRI system. As described in sub-process 195, this AIMD patient implant data may be downloaded to and stored by the AIMD 300 even prior to operation of the AIMD 300 with the MRI system. Using the actually installed configuration data of the AIMD 300 within a particular patient 360 enables a unique level of compatibility to be defined between that particular installed AIMD 300 and the MRI system. Accuracy and precision in the calculation of the level of compatibility may thus be enhanced.

While the invention has been described in connection with what is presently considered to be practical exemplary embodiments, it is to be understood that the invention is not limited to the disclosed embodiments, but, on the contrary, is intended to cover all variations, modifications and equivalent arrangements included within the spirit and scope of the appended claims.

What is claimed is:

1. A system comprising:
a MRI system; and
an AIMD communications interface, operably coupled to the MRI system, that communicates information with an AIMD having a lead, the communicated information including data relating to a configuration of the lead in a patient.

2. The system as in claim 1 wherein the lead of the AIMD defines a loop area, and the data relating to the configuration of the lead in the patient includes at least one of a size or position of the loop area.

3. The system as in claim 1 wherein the data relating to the configuration of the lead in the patient includes data corresponding to a pictorial representation of the lead and at least part of a body of the patient.

4. The system as in claim 1 wherein the communicated information received by the AIMD communications interface from the AIMD includes information indicating a limit on at least one operational parameter of the MRI machine determined by the AIMD based on the data relating to the configuration of the lead in the patient.

5. The system as in claim 1 wherein the MRI machine determines a limit on at least one operational parameter of the MRI machine based on the data relating to the configuration of the lead in the patient.

6. The system as in claim 4 wherein the limit relates to a maximum limit on at least one of a strength level of a magnetic field or a radio-frequency (RF) field of the MRI machine.

7. The system as in claim 5 wherein the limit relates to a maximum limit on at least one of a strength level of a magnetic field or a radio-frequency (RF) field of the MRI machine.

8. A method of operating a system comprising a MRI machine and an AIMD communications interface disposed to communicate with an AIMD having a lead, the method comprising:
communicating information with the AIMD;
processing information received from the AIMD as at least part of the communicated information, the communicated information including data relating to a configuration of the lead in a patient.

9. The method as in claim 8 wherein the lead of the AIMD defines a loop area, and the data relating to the configuration of the lead in the patient includes at least one of a size or position of the loop area.

10. The method as in claim 8 wherein the data relating to the configuration of the lead in the patient includes data corresponding to a pictorial representation of the lead and at least part of a body of the patient.

11. The method as in claim 8 wherein the communicated information received from the AIMD includes information indicating a limit on at least one operational parameter of the MRI machine determined by the AIMD based on the data relating to the configuration of the lead in the patient.

12. The method as in claim 8 wherein the MRI machine determines a limit on at least one operational parameter of the MRI machine based on the data relating to the configuration of the lead in the patient.

13. The method as in claim 11 wherein the limit relates to a maximum limit on at least one of a strength level of a magnetic field or a radio-frequency (RF) field of the MRI machine.

14. The method as in claim 12 wherein the limit relates to a maximum limit on at least one of a strength level of a magnetic field or a radio-frequency (RF) field of the MRI machine.

15. An active implanted medical device (AIMD) having at least one lead for use with a magnetic resonance imaging (MRI) machine, the AIMD comprising:
an integrated circuit that processes data relating to a configuration of the lead in a patient; and
a communications interface, operably coupled to the integrated circuit, that communicates information with the MRI machine, the communicated information including at least one of the data relating to a configuration of the lead in the patient or a limit on at least one operational parameter of the MRI machine determined based on the data relating to the configuration of the lead in the patient.

16. The AIMD as in claim 15 wherein the lead of the AIMD defines a loop area, and the data relating to the configuration of the lead in the patient includes at least one of a size or position of the loop area.

17. The AIMD as in claim 15 wherein the data relating to the configuration of the lead in the patient includes data corresponding to a pictorial representation of the lead and at least part of a body of the patient.

18. The AIMD as in claim 15 wherein the communicated information includes data relating to the limit, the limit being determined by the integrated circuit of the AIMD based on the data relating to the configuration of the lead in the patient and communicated information received from the MRI machine.

19. The AIMD as in claim 15 wherein the communicated information received by the communications interface of the AIMD from the MRI machine includes information indicating the limit, the limit being determined by the MRI machine based on the data relating to the configuration of the lead in the patient.

20. The AIMD as in claim 18 wherein the limit relates to a maximum limit on at least one of a strength level of a magnetic field or a radio-frequency (RF) field of the MRI machine.
